(19) United States
(12) Patent Application Publication
(10) Pub. No.: US 2026/0365998 A1
(43) Pub. Date: Nov. 19, 2026

(54) Method for improving productivity of biosynthetic superoxide dismutase (SOD)

(71) Applicant: Beijing Active Blue Crystal Biotechnology Co., Ltd., Beijing (CN)

(72) Inventors: Feng LING, Beijing (CN); Wenyong Ding, Beijing (CN); Yanxia Song, Beijing (CN)

(73) Assignee: Beijing Active Blue Crystal Biotechnology Co., Ltd., Beijing (CN)

(21) Appl. No.: 19/154,972

(22) PCT Filed: Jan. 4, 2024
(86) PCT No.: PCT/CN2024/070483

§ 371 Date: Apr. 28, 2026

(30) Foreign Application Priority Data
Feb. 9, 2023 (CN) 20231008622.9

(51) Int. Cl. C12N 9/02; C12N 1/165; C12N 1/185; C12P 21/02; C12R 1/84; C12R 1/865

(52) U.S. Cl. CPC C12N 9/0089; C12N 1/165; C12N 1/185; C12P 21/02; C12R 2001/84; C12R 2001/865; C12Y 115/01001

(57) ABSTRACT

The present invention discloses a method for Increasing the productivity of biosynthetic superoxide dismutase (SOD). In the initial stage, a high yeast inoculation amount is used, combined with the addition of carbon sources, nitrogen sources and nutrients required for biosynthesis, and by increasing the dissolved oxygen value in the culture medium, using the optimal temperature and pH value, so that the yeast content in the culture medium increases rapidly in the middle stage of biosynthesis. After the middle stage of biosynthesis, the method of continuously adding carbon sources and nitrogen sources is adopted to continuously provide the biosynthetic superoxide dismutase (SOD) with the energy substances required for yeast biosynthesis. Combined with increasing the dissolved oxygen value in the culture medium and continuously adding copper and zinc salts to the fermentation tank, the yeast is stimulated to quickly synthesize copper and zinc superoxide dismutase (SOD) in the body. Through the above method, the yeast content and the superoxide dismutase (SOD) content in the yeast are increased, and the overall biosynthesis time is shortened. ultimately increasing the productivity of superoxide dismutase (SOD) biosynthesis. The present invention discloses a method for increasing the productivity of biosynthetic superoxide dismutase (SOD). In the initial stage, a high yeast inoculation amount is used, combined with the addition of carbon sources, nitrogen sources and nutrients required for biosynthesis, and by increasing the dissolved oxygen value in the culture medium, using the optimal temperature and pH value, so that the yeast content in the culture medium increases rapidly in the middle stage of biosynthesis. After the middle stage of biosynthesis, the method of continuously adding carbon sources and nitrogen sources is adopted to continuously provide the biosynthetic superoxide dismutase (SOD) with the energy substances required for yeast biosynthesis. Combined with increasing the dissolved oxygen value in the culture medium and continuously adding copper and zinc salts to the fermentation tank, the yeast is stimulated to quickly synthesize copper and zinc superoxide dismutase (SOD) in the body. Through the above method, the yeast content and the superoxide dismutase (SOD) content in the yeast are increased, and the overall biosynthesis time is shortened, ultimately increasing the productivity of superoxide dismutase (SOD) biosynthesis.

### CROSS REFERENCE TO RELATED APPLICATIONS

This application is a U.S.C. 371 United States national stage of International Application No. PCT/CN24/70483, filed Jan. 4, 2024, which claims priority to Chinese patent application no. 202310086222.9, filed Feb. 9, 2023, the full disclosure of which is incorporated herein by reference.

### FIELD OF THE INVENTION

The present invention belongs to the field of biosynthesis technology, and more specifically relates to a method for increasing the productivity of biosynthetic superoxide dismutase (SOD).

### BACKGROUND OF THE INVENTION

Superoxide dismutase (SOD) is an important member of the antioxidant enzyme system in the biological system. It is widely present in microorganisms, plants and animals. It is an antioxidant metalloenzyme that can catalyze the dismutation of superoxide anion free radicals to generate oxygen and hydrogen peroxide. It plays a vital role in the body's oxidation and antioxidant balance and is inseparable from the occurrence and development of many diseases.

The main function of superoxide dismutase (SOD) is to catalyze the dismutation of superoxide anion free radicals into hydrogen peroxide and oxygen. Superoxide anions are the product of normal metabolism in organisms, but the accumulation of free radicals will cause lipid peroxidation of cell membranes and cause membrane fission, leading to cell damage and even death.

Superoxide dismutase (SOD) is the most important and best free radical scavenger in cells to maintain the body's metabolic balance. Superoxide dismutase is a type of metalloenzyme, which can be divided into three types:
The first type contains copper and zinc, called copper-zinc superoxide dismutase, which is blue-green and mainly exists in the cytoplasm of eukaryotic cells;
The second type contains manganese, called manganese-containing superoxide dismutase, which is purple and mainly exists in prokaryotic cells and eukaryotic cells matrix (such as mitochondria, etc.);
The third type contains iron, called iron-containing superoxide dismutase, which is yellow-brown and mainly exists in prokaryotic cells and a few plant cells.

In eukaryotic organisms, including humans, the main superoxide dismutase is the first type of copper-zinc superoxide dismutase. The superoxide dismutase biosynthesized according to the present disclosure belongs to the first type, namely copper-zinc superoxide dismutase.

Superoxide dismutase (SOD) can play an anti-oxidation and anti-aging role:

Superoxide dismutase (SOD) can remove and reduce excessive free radicals in the human body and delay aging. It can continuously remove oxygen free radicals generated by excessive oxygen consumption of melanin due to light exposure, prevent the formation of excessive melanin, and also continuously remove oxygen free radicals generated by lipofuscin and waxy fat to avoid excessive lipid peroxidation, and reduce the formation of lipofuscin and waxy fat, thereby playing an anti-aging role.

Superoxide dismutase (SOD) can play an anti-tumor role: In the human body, superoxide dismutase (SOD) can effectively inhibit cancer cells by removing superoxide anion free radicals. The superoxide dismutase (SOD) in the human body tends to decline with age, Its decline leads to a decline in the body's antioxidant capacity, and then a decline in resistance. In severe cases, tumors may occur. Therefore, the elderly people are generally more likely to be patients with tumors. From this perspective, detecting the content of superoxide dismutase (SOD) in a person's body can be used as an important indicator to determine whether a person has cancer and the aging index.

Anti-inflammatory effect: Superoxide dismutase (SOD) has a strong anti-inflammatory effect. Using superoxide dismutase (SOD) to treat pleurisy can reduce the accumulation of pleural effusion and white blood cells. Superoxide dismutase (SOD) can also inhibit arthritis, reduce the precipitation of IgG in the glomeruli, and interfere with the accumulation of white blood cells in the inflammatory site of the glomeruli. Superoxide dismutase (SOD) can inhibit the changes in organ permeability caused by acute tracheitis which caused by xanthine/xanthine oxidase, and reduce the accumulation of polymorphonuclear leukocytes in the lungs. Superoxide dismutase (SOD) has a therapeutic and improving effect on idiopathic proctitis. Injecting an appropriate amount of superoxide dismutase (SOD) can improve the appearance of joints, improve joint mobility, and effectively treat osteoarthritis. It can be seen that superoxide dismutase (SOD) has a good application prospect for the treatment of inflammation.

Application fields of superoxide dismutase (SOD):

Medicine: Superoxide free radicals in the body can cause various diseases. Superoxide dismutase (SOD) is its natural scavenger and maintains a dynamic balance with it under normal circumstances. However, under pathological conditions, excessive superoxide free radicals are produced. The superoxide dismutase (SOD) produced by the body itself cannot completely remove these excessive superoxide free radicals, which are harmful to the body. Superoxide dismutase (SOD) can catalyze their dismutation reaction and alleviate the condition.

Food industry: Studies have shown that adding superoxide dismutase (SOD) to food as a natural antioxidant can be used as a preservative.

Clinical trials have shown that oral superoxide dismutase (SOD) is effective and can be absorbed into the human body through the gastrointestinal tract. Therefore, superoxide dismutase (SOD) chewing gum, superoxide dismutase (SOD) lozenges and oral liquid have appeared one after another, becoming one of the important ways to supplement superoxide dismutase (SOD) in vitro.

Daily chemical industry: The application of superoxide dismutase (SOD) in daily chemical industry is mainly skin care products and toothpaste.

Superoxide dismutase (SOD) has the following main functions in skin care products: First, as a cosmetic additive, it prevents skin aging, reduces wrinkles and removes spots, and has a skin care effect; second, it prevents and treats related skin diseases, such as dermatitis, acne, and skin burns.

Since superoxide dismutase (SOD) has an anti-inflammatory effect and is pure natural with no toxic side effects, it plays an important role in oral and dental care.

Agriculture: Overexpression of superoxide dismutase (SOD) in transgenic plants can improve the resistance of plants to adverse environments to varying degrees. By genetic engineering, increasing the expression of superoxide dismutase (SOD) in plants can greatly enhance the stress resistance of plants.

From the above introduction, it can be seen that superoxide dismutase (SOD) is important to human health and life, so it is very important to find a high-content, low-cost method for producing superoxide dismutase (SOD), especially the method for producing the first type superoxide dismutase (copper-zinc superoxide dismutase) (SOD).

There are four main methods for producing superoxide dismutase (SOD):
1. Extraction method using animal blood;
2. Extraction from plants;
3. Production by microbial biosynthesis;
4. Production of superoxide dismutase (SOD) by genetic engineering method.

Using animal blood extraction method: Due to the limitation of raw materials, it cannot be mass-produced, and there will be viruses in animal blood, which cannot be directly used for human body, and the cost is too high. Due to the above shortcomings, it cannot be industrialized and mass-produced.

Extraction from plants: Due to the limitation of raw materials and the low content of superoxide dismutase (SOD) in natural plants, it is impossible to obtain high-purity superoxide dismutase (SOD), which limits the application of superoxide dismutase (SOD) in many fields.

Production of superoxide dismutase (SOD) by genetic engineering method has only been studied in recent years. The existing technology can only produce small batches, and the cost is too high, and it cannot meet the requirements of large-scale production.

The microbial biosynthesis method is the subject of the presently disclosed biosynthesis method. The microorganisms used for the production of superoxide dismutase (SOD) by microbial biosynthesis are mainly yeast and bacteria, according to the production method of the present invention. The advantages of this method are that there is no restriction on raw materials, and the biosynthesis process can be strictly controlled to perform aseptic operation, so the produced superoxide dismutase (SOD) does not contain any harmful viruses, bacteria and other pathogens.

In the prior art, the main reason for limiting the industrial production of superoxide dismutase (SOD) biosynthesized by microorganisms (mainly yeast) is that the productivity of superoxide dismutase biosynthesized by yeast is too low, that is, the biological activity (U) of superoxide dismutase biosynthesized per unit volume of fermentation tank (liter) per unit time (hour) is too low.

In the prior art, the main problem is that at the end of biosynthesis, the yeast content in the biosynthesis liquid is too low, the superoxide dismutase (SOD) content in the yeast is too low, and the biosynthesis time is too long, that is, the productivity is too low, which makes the equipment investment too large, the raw and auxiliary materials consumption is too high, and the production cost of superoxide dismutase (SOD) is too high. In addition, since the content of biosynthesized superoxide dismutase (SOD) in yeast is too low, it causes difficulties in the later purification process and cannot obtain high-purity superoxide dismutase (SOD).

Therefore, finding a biosynthesis method with high superoxide dismutase (SOD) content, high yeast content and short time, that is, a biosynthesis method with high productivity, is the key to realizing the industrialized large-scale production of superoxide dismutase (SOD).

### SUMMARY OF THE INVENTION

The object of the present invention is to find a biosynthesis method to produce superoxide dismutase with high superoxide dismutase (SOD) content, high yeast content and short time, that is, a biosynthesis method to produce superoxide dismutase with high productivity.

The object of the present invention is achieved by the following technical scheme:

In a first embodiment, a method for increasing the productivity of biosynthetic superoxide dismutase (SOD) is provided, specifically comprising the following steps:
(1) Adding water to a fermentation tank, and then adding the carbon source, nitrogen source and nutrient salts required for yeast biosynthesis of superoxide dismutase (SOD) to obtain a basic culture medium;
(2) Using a high yeast inoculation amount in the basic culture medium;
(3) Setting the air flow rate of the fermentation tank, stirring speed, temperature and pH value, start the biosynthesis of superoxide dismutase (SOD);
(4) When the initial stage of biosynthesis is reached, start to add carbon source and nitrogen source to the fermentation tank continuously;
(5) When the middle stage of biosynthesis is reached, start to add copper and zinc salts to the fermentation tank continuously, and at the same time increase the air flow rate and stirring speed;
(6) After the biosynthesis is completed, yeast with high yeast content and high superoxide dismutase (SOD) biological activity content in the yeast is obtained in the biosynthesis liquid.

In some aspects, the yeast is selected from Saccharomyces cerevisiae, Pichia pastoris, etc.

In some aspects, the high yeast inoculation amount is higher than about 10 g/L.

In some aspects, the carbon source described includes glucose, sucrose and/or other carbon sources.

In some aspects, the nitrogen sources include peptone, yeast powder, urea, ammonium sulfate and/or other nitrogen sources.

In some aspects, the nutrient salts include copper sulfate, zinc sulfate, dipotassium hydrogen phosphate, potassium dihydrogen phosphate, magnesium sulfate, calcium chloride, sodium chloride, and/or other nutrient salts.

In some aspects, the air flow rate is about 1-1.5 vvm (volumetric gas-toliquid ratio; i.e., air volume fed into fermentation tank per minute/biosynthetic liquid volume).

In some aspects, the stirring speed in step (3) is about 200-500 rpm.

In some aspects, the temperature is within the range of about 28-36 °C.

In some aspects, the pH value is within the range of about 4.5-7.5.

In some aspects, the time of the initial stage of biosynthesis described is about 2-6 hours.

In some aspects, the carbon source and nitrogen source are added until 5-10 hours before the end of biosynthesis.

In some aspects, the copper and zinc salts include copper sulfate, zinc sulfate, and/or other copper and zinc salts.

In some aspects, the time of the middle stage of biosynthesis is about 20-24 hours.

In some aspects, when the biosynthesis reaches the middle stage of biosynthesis, the wet yeast content in the biosynthesis liquid is not less than about 170 g/L, and the dry matter content of the wet yeast is calculated as about 33%.

In some aspects, the nutrient salt is added until about 3-6 hours before the end of biosynthesis.

In some aspects, the air flow rate is about 1.5-2.5 vvm (air volume fed into the fermentation take per minute/volume of biosynthesis liquid).

In some aspects, the stirring speed in step (5) is about 500-800 rpm.

In some aspects, the biosynthesis completion time in step (6) is not more than about 55 hours.

In some aspects, the yeast content in the biosynthesis liquid in step (6) is not less than about 200 g/L, calculated as the wet yeast content per liter of biosynthesis liquid, and the dry matter content of the wet yeast is calculated as about 33%.

In some aspects, the superoxide dismutase (SOD) biological activity content in step (6) is not less than about 8000 U/g wet yeast, and the dry matter content of the wet yeast is calculated as about 33%.

In some aspects, the superoxide dismutase (SOD) biological activity in step (6) is determined by the pyrogallol auto-oxidation method.

In some aspects, the superoxide dismutase (SOD) described in step (6) is an intracellular enzyme.

The superoxide dismutase (SOD) described in step (6) is a Cu/Zn superoxide dismutase (SOD).

The advantages and beneficial effects of the present invention include:

First, the present invention belongs to a biosynthesis technology. Biosynthesis is a multi-step, enzyme-catalyzed process.

In this process, the substrate which include the carbon source, nitrogen source and nutrient salts is converted into more complex substances in the organelle. In the biosynthesis process, simple compounds are changed, converted into other compounds, or combined into macromolecular compounds. This process often includes metabolic pathways. These metabolic pathways may occur in one organelle, and some may involve several organelles. The superoxide dismutase described in the present invention is synthesized in yeast cells in this way.

Second, the present invention adopts a high yeast inoculation amount in the initial stage of biosynthesis, and the high yeast inoculation amount is combined with the addition of appropriate carbon sources, nitrogen sources and nutrients required for biosynthesis, and the dissolved oxygen value in the culture medium is increased by high air intake and high stirring speed, and the optimal temperature and pH value are adopted, so that the yeast content in the culture medium reaches 170 g/L or more in the middle stage of biosynthesis.

Third, in the middle stage of biosynthesis, that is, on the basis of the yeast content in the culture medium reaching 170 g/L or more, the present invention adopts a method of continuing to add carbon sources and nitrogen sources, combined with a method of increasing air flow rate and stirring speed to further increase the dissolved oxygen value in the culture medium, to provide the necessary energy substances and oxygen for the continued growth of yeast, and finally the yeast content in the biosynthesis liquid is increased, so that the yeast content in the final biosynthesis liquid exceeds 200 g/L.

Fourth, in the middle stage of biosynthesis, when the content of yeast in the culture medium reaches 170 g/L or more, the present invention adopts a method of continuously adding carbon source and nitrogen source to continuously provide the energy substances required for the biosynthesis of yeast superoxide dismutase (SOD), and further increases the dissolved oxygen value in the culture medium by increasing the air flow rate and the stirring speed to provide the oxygen required for yeast biosynthesis. At the same time, by continuously adding copper and zinc salts to the fermentation tank, the yeast is stimulated to rapidly synthesize Cu/Zn superoxide dismutase (SOD) in vivo, and finally the biological activity content of superoxide dismutase (SOD) in the yeast in the biosynthesis liquid is increased, and the biological activity content of superoxide dismutase (SOD) in the yeast exceeds 8000 U/g Wet yeast.

Fifth, the present invention uses the above method to rapidly increase the yeast content in the culture medium, and the biological activity content of superoxide dismutase (SOD) in the yeast is also rapidly increased, and ultimately shortens the overall biosynthesis time, so that the biosynthesis completion time does not exceed 55 hours.

Sixth, the present invention uses a method of rapidly increasing the yeast content in the culture medium and rapidly increasing the biological activity content of superoxide dismutase (SOD) in the yeast to shorten the overall biosynthesis time, and ultimately increases the productivity of biosynthetic superoxide dismutase (SOD).

### EXAMPLES

The present invention will be further described below through specific examples.

### Example 1

A 3-liter fully automatic fermentation tank was used, which has automatic control functions for temperature, pH and dissolved oxygen value (stirring speed and air flow rate), and has a 6-way feeding automatic control system.
(1) 70 g/L of sterilized sugar, 1 g/L of peptone, 2 g/L of yeast powder, 1.3 g/L of urea, 0.03 g/L of copper sulfate pentahydrate, 0.03 g/L of zinc sulfate heptahydrate, 1.5 g/L of dipotassium hydrogen phosphate, 3 g/L of potassium dihydrogen phosphate, and 0.3 g/L of magnesium sulfate heptahydrate were added to the fermentation tank, and then ultrapure water was added to 2.5 liters to obtain a basic culture medium.
(2) Angel brewer's yeast (fresh and dry yeast) was used, and the yeast inoculation amount was 10 g/L.
(3) The air flow rate of the fermentation tank was set to 1 v/v/m (air volume fed into the fermentation tank per minute/volume of fermentation tank), the stirring speed to 200 rpm, the temperature to 30 °C and the pH value to 6.5, and the biosynthesis of superoxide dismutase (SOD) was started.
(4) After 2 hours of biosynthesis, glucose solution 7 ml/h (glucose content 50%), yeast powder solution 0.6 ml/h (yeast powder content 20%), urea solution 0.4 ml/h (urea content 20%) were added, and the addition was stopped after 45 hours.
(5) After 20 hours of biosynthesis, copper sulfate pentahydrate solution 0.01 ml/h (copper sulfate content 15%) and zinc sulfate heptahydrate solution 0.01 ml/h (zinc sulfate content 15%) were added, the air flow rate was increased to 1.5 vvm, the stirring speed was increased to 500 rpm, and the addition was stopped after 45 hours.
(6) Biosynthesis was terminated after 50 hours, and 2.6 L of biosynthesis liquid was obtained.
(7) After the biosynthesis reached 20 hours, 50 ml of the biosynthesis liquid was taken and separated with a separator. The separator was set to 6000 rpm, the separation factor was 6439, and the separation time was 20 minutes. Through separation, wet yeast is obtained. The obtained wet yeast was dissolved in 5 times the volume of water, stirred evenly and separated again. The above steps were repeated three times to obtain pure wet yeast. The weight of the pure wet yeast was 8.6 g. The content of pure wet yeast in the biosynthesis liquid was calculated to be 17.2% (w/v).
(8) 500 ml of the biosynthesis liquid after the biosynthesis is completed was taken, and the biosynthesis liquid was separated with a separator. The separator was set to 6000 rpm, the separation factor was 6439, and the separation time was 20 minutes. After separation, wet yeast was obtained. The obtained wet yeast was dissolved in 5 times the volume of water, stirred evenly and separated again. The above steps were repeated three times to obtain pure wet yeast. The weight of the pure wet yeast was 104 g. The content of the pure wet yeast in the biosynthesis liquid was calculated to be 20.8% (w/v).
(9) 100 g of the pure wet yeast obtained in step (8) was taken, and then a homogenizer was used to break the yeast wall. The pressure of the homogenizer was set to 140 MPa, the flow rate was set to 50 ml/min, and the number of cycles was 5 times to obtain a yeast wall-breaking liquid. The yeast wall-breaking liquid was tested by the pyrogallol auto-oxidation method. The superoxide dismutase (SOD) content in the wet yeast was 8200 U/g After calculation, the productivity of superoxide dismutase synthesized by yeast was 29564 U/L/h {Superoxide dismutase (U) synthesized by the unit volume of fermentation tank (liter) per unit time (hour)}.

### Example 2

A 3-liter fully automatic fermentation tank was used, which has automatic control functions for temperature, pH and dissolved oxygen value (stirring speed and air flow rate), and has a 6-way feeding automatic control system.
(1) 75 g/L of sterilized sugar, 1.2 g/L of peptone, 2.3 g/L of yeast powder, 1.2 g/L of urea, 0.04 g/L of copper sulfate pentahydrate, 0.04 g/L of zinc sulfate heptahydrate, 1.7 g/L of dipotassium hydrogen phosphate, 4 g/L of potassium dihydrogen phosphate, and 0.4 g/L of magnesium sulfate heptahydrate was added to the fermentation tank, and then ultrapure water was added to 2.5 liters to obtain a basic culture medium.
(2) Angel brewer's yeast (fresh dry yeast) was used, and the yeast inoculation amount was 12 g/L.
(3) The air flow rate of the fermentation tank was set to 1.2 v/v/m (air volume fed to the fermentation tank per minute/volume of fermentation tank), the stirring speed was 300 rpm, the temperature was 31 °C and the pH value was 6.8, and the biosynthesis of superoxide dismutase (SOD) was started.
(4) After 3 hours of biosynthesis, glucose solution was added at 8 ml/h (glucose content was 50%), peptone solution at 0.3 ml/h (peptone content was 20%), yeast powder solution at 0.6 ml/h (yeast powder content was 20%), and urea solution at 0.3 ml/h (urea content was 20%), and the addition was stopped after 45 hours.
(5) After 24 hours of biosynthesis, 0.02 ml/h of copper sulfate pentahydrate solution (copper sulfate content is 15%) and 0.02 ml/h of zinc sulfate heptahydrate solution (zinc sulfate content is 15%) were added, the air flow rate was increased to 2 v/v/m, the speed was increased to 700 rpm, and the addition was stopped after 46 hours.
(6) Biosynthesis was completed after 55 hours, and 2.65 L of biosynthesis liquid was obtained.
(7) After the biosynthesis reached 24 hours, 50 ml of the biosynthesis liquid was taken and separated with a separator. The separator was set to 6000 rpm, the separation factor was 6439, and the separation time was 20 minutes. Through separation, wet yeast was obtained. The obtained wet yeast was dissolved in 5 times the volume of water, stirred evenly and separated again. The above steps were repeated three times to obtain pure wet yeast. The weight of the pure wet yeast was 8.9 g. The content of pure wet yeast in the biosynthesis liquid was calculated to be 17.8% (w/v).
(8) 500 ml of the biosynthesis liquid was taken after the biosynthesis was completed, and the biosynthesis liquid was separated with a separator. The separator was set to 6000 rpm, the separation factor was 6439, and the separation time was 20 minutes. Through separation, wet yeast was obtained. The obtained wet yeast was dissolved in 5 times the volume of water, stirred evenly and separated again. The above steps were repeated three times to obtain pure wet yeast. The weight of the pure wet yeast was 112 g. The content of the pure wet yeast in the biosynthesis liquid was calculated to be 22.4% (w/v).
(9) 100 g of the pure wet yeast obtained in step (8) was taken, and a homogenizer was then used to break the yeast wall. The homogenizer pressure was set to 140 MPa, the flow rate was set to 50 ml/min, and the number of cycles was 5 times to obtain a yeast wall-breaking liquid. The yeast wall-breaking liquid was tested by the pyrogallol auto-oxidation method. The superoxide dismutase (SOD) content in the wet yeast was 9100U/g.

After calculation, the productivity of superoxide dismutase biosynthesized by yeast was 32738 U/L/h {superoxide dismutase (U) biosynthesized per unit volume of fermentation tank (liter) per unit time (hour)}.

### Example 3

A 3-liter fully automatic fermentation tank was used, which has automatic control functions for temperature, pH and dissolved oxygen value (stirring speed and air flow rate), and has a 6-way feeding automatic control system.
(1) 80 g/L of sterilized sugar, 1.5 g/L of peptone, 2.5 g/L of yeast powder, 1.25 g/L of urea, 0.05 g/L of copper sulfate pentahydrate, 0.05 g/L of zinc sulfate heptahydrate, 2 g/L of dipotassium hydrogen phosphate, 5 g/L of potassium dihydrogen phosphate, and 0.5 g/L of magnesium sulfate heptahydrate were added to the fermentation tank, and then ultrapure water added to 2.5 liters to obtain a basic culture medium.
(2) Angel brewer's yeast (fresh and dry yeast) was used, and the yeast inoculation amount was 15 g/L.
(3) The air flow rate of the fermenter was set to 1.5 v/v/m (air volume fed into the fermentation tank per minute/the volume of fermentation tank), the stirring speed to 500 rpm, the temperature to 32 °C and the pH value to 7, and biosynthesis of superoxide dismutase (SOD) was started.
(4) After 4 hours of biosynthesis, started to add glucose solution 10 ml/h (glucose content is 50%), peptone solution 0.4 ml/h (peptone content is 20%), yeast powder solution 0.7 ml/h (yeast powder content is 20%), urea solution 0.36 ml/h (urea content is 20%), and stopped the addition after 48 hours.
(5) After 22 hours of biosynthesis, 0.03 ml/h of copper sulfate pentahydrate solution (copper sulfate content is 15%) and 0.03 ml/h of zinc sulfate heptahydrate solution (zinc sulfate content is 15%) were added, the air flow rate was increased to 2.5 v/v/m/, the stirring speed was increased to 800 rpm, and the addition was stopped after 50 hours.
(6) The biosynthesis was terminated after 52 hours, and 2.7 L of biosynthesis liquid was obtained.
(7) After the biosynthesis reached 22 hours, 50 ml of the biosynthesis liquid was taken and separated with a separator. The separator was set to 6000 rpm, the separation factor was 6439, and the separation time was 20 minutes. Through separation, wet yeast was obtained. The obtained wet yeast was dissolved in 5 times the volume of water, stirred evenly and separated again. The above steps were repeated three times to obtain pure wet yeast. The weight of the pure wet yeast was 9.2 g. The content of pure wet yeast in the biosynthesis liquid was calculated to be 18.4% (w/v).
(8) 500 ml of the biosynthesis liquid was taken after the biosynthesis is completed, and the biosynthesis liquid was separated with a separator. The separator was set to 6000 rpm, the separation factor was 6439, and the separation time was 20 minutes. After separation, wet yeast was obtained. The obtained wet yeast was dissolved in 5 times the volume of water, stirred evenly and separated again. The above steps were repeated three times to obtain pure wet yeast. The weight of the pure wet yeast was 118 g. The content of the pure wet yeast in the biosynthesis liquid was calculated to be 23.6% (w/v).
(9) 100 g of the pure wet yeast obtained in step (8) was taken, and then a homogenizer to break the yeast cell wall. The homogenizer pressure was set to 140 MPa, the flow rate was set to 50 ml/min, and the number of cycles was 5 times to obtain a yeast cell wall liquid. The yeast cell wall liquid was tested by pyrogallol auto-oxidation method. The superoxide dismutase (SOD) content in the wet yeast was 9800 U/g.

After calculation, the productivity of superoxide dismutase biosynthesized by yeast was 40029 U/L/h {superoxide dismutase (U) biosynthesized per unit volume of fermentation tank (liter) per unit time (hour)}.

1-16. (canceled)

17. A method for increasing the productivity of biosynthetic superoxide dismutase (SOD), comprising the following steps:
(1) adding sterilized sugar 70 g/L, peptone 1 g/L, yeast powder 2 g/L, urea 1.3 g/L, copper sulfate pentahydrate 0.03 g/L, zinc sulfate heptahydrate 0.03 g/L, dipotassium hydrogen phosphate 1.5 g/L, potassium dihydrogen phosphate 3 g/L, magnesium sulfate heptahydrate 0.3 g/L to a fermentation tank, and then adding ultrapure water to 2.5 liters to obtain a basic medium, and
(2) inoculating the basic medium with a high yeast inoculation concentration of 10-15 g/L, and
(3) setting the air intake volume of the fermentation tank to 1-1.5 v/v/m, wherein v/v/m corresponds to volume of air intake per minute/volume of the biosynthetic tank, the stirring speed to 200-500 rpm, the temperature to 28 °C-36 °C, and the pH value to 4.5-7.5, and initiating the biosynthesis of superoxide dismutase (SOD), and
(4) when the initial stage of biosynthesis is reached, which lasts for 2-6 hours, starting to continuously add carbon sources to the fermentation tank: glucose solution at 7 ml/h, with a glucose content of 50% in the glucose solution; and nitrogen sources: yeast powder solution at 0.6 ml/h, with a yeast powder content of 20% in the yeast powder solution, and urea solution at 0.4 ml/h, with a urea content of 20% in the urea solution and continue adding for 40-45 hours, and
(5) when reaching the middle stage of biosynthesis, which lasts for 20-24 hours, starting to continuously add 0.01 ml/h of copper sulfate pentahydrate solution, with a copper sulfate content of 15%, and 0.01 ml/h of zinc sulfate heptahydrate solution, with a zinc sulfate content of 15%, increase the air flow rate to 1.5 vvm, the rotation speed to 500 rpm, and continue adding for 44-47 hours before stopping, and
(6) after the end of biosynthesis, the end time of biosynthesis being 50-55 hours; obtaining the high yeast content: 20-20.8% (w/v); and the high superoxide dismutase (SOD) biological activity content is 8000-8200 U/g; the production rate of yeast biosynthetic superoxide dismutase is 29564 U/L/h; wherein the calculation of high yeast content obtained in the biosynthetic liquid is based on the weight ratio (w/v) of pure wet yeast in the volume of biosynthetic liquid; the biological activity content of superoxide dismutase (SOD) is calculated based on the activity content of superoxide dismutase (SOD) in wet yeast, and the dry matter content in wet yeast is calculated at 33%, with the determination method by pyrogallol method; the production rate of yeast biosynthetic superoxide dismutase is calculated as the superoxide dismutase (U) biosynthesized per unit volume of fermentation tank (liter) per unit time (hour).

18. The method according to claim 17, wherein in step (1), the yeast comprises Saccharomyces cerevisiae and Pichia pastoris.

19. The method according to claim 17, wherein in step (5), the time of the biosynthetic process reaches the mid-phase, the wet yeast content in the biosynthetic solution is not less than 170 g/L, and the dry matter content of the wet yeast is calculated at 33%.

20. The method according to claim 1, wherein in step (6), the superoxide dismutase (SOD) is an intracellular enzyme, and the superoxide dismutase (SOD) is a copper- and zinc-containing superoxide dismutase (SOD).

21. A method for enhancing the production rate of biosynthetic superoxide dismutase (SOD), characterized by the following steps:
(1) adding sterilized sugar 75 g/L, peptone 1.2 g/L, yeast powder 2.3 g/L, urea 1.2 g/L, copper sulfate pentahydrate 0.04 g/L, zinc sulfate heptahydrate 0.04 g/L, dipotassium hydrogen phosphate 1.7 g/L, potassium dihydrogen phosphate 4 g/L, magnesium sulfate heptahydrate 0.4 g/L to a fermentation tank, and then adding ultrapure water to 2.5 liters to obtain a basic medium, and
(2) inoculating the basic medium with a high yeast inoculum concentration of 10-15 g/L, and
(3) setting the air intake volume of the fermentation tank to 1-1.5 v/v/m, the stirring speed to 200-500 rpm, the temperature to 28-36 °C, and the pH value to 4.5-7.5, and initiating the biosynthesis of superoxide dismutase (SOD), and
(4) when reach the initial stage of biosynthesis, which lasts for 2-6 hours, starting to continuously add carbon sources to the fermentation tank: glucose solution at 8 ml/h, with a glucose content of 50% in the glucose solution; and nitrogen sources: yeast powder solution at 0.6 ml/h, with a yeast powder content of 20% in the yeast powder solution, nitrogen peptone solution at 0.3 ml/h, with a nitrogen peptone content of 20% in the nitrogen peptone solution, and urea solution at 0.3 ml/h, with a urea content of 20% in the urea solution, continue adding for 45-50 hours, and
(5) when reaching the middle stage of biosynthesis, which lasts for 20-24 hours, starting to continuously add 0.02 ml/h of copper sulfate pentahydrate solution, with a copper sulfate content of 15%. Add 0.02 ml/h of zinc sulfate heptahydrate solution, with a zinc sulfate content of 15%, increasing the air flow rate to 2 vvm, the rotation speed to 700 rpm, and continue adding for 49-52 hours before stopping, and
(6) after the biosynthesis is completed, the biosynthesis completion time being 55 hours; obtaining the high yeast content in the biosynthesis solution of 20-22.4% (w/v); and the high superoxide dismutase (SOD) biological activity content of 8000-9100 U/g; the production rate of yeast biosynthetic superoxide dismutase being 32738 U/L/h; wherein the calculation of high yeast content obtained in the biosynthesis solution is based on the weight ratio (w/v) of pure wet yeast in the volume of the biosynthesis solution; the biological activity content of superoxide dismutase (SOD) is calculated based on the activity content of superoxide dismutase (SOD) in wet yeast, and the dry matter content in wet yeast is calculated at 33%, The determination method is based on the pyrogallol method; the production rate of yeast biosynthetic superoxide dismutase is calculated as the amount of superoxide dismutase (U) biosynthesized per unit volume of fermentation tank (liter) per unit time (hour).

22. The method according to claim 21, wherein in step (1), the yeast comprises Saccharomyces cerevisiae and Pichia pastoris.

23. The method according to claim 21, wherein in step (5), when the biosynthesis reaches the mid-stage, the wet yeast content in the biosynthetic solution is not less than 170 g/L, and the dry matter content of the wet yeast is calculated at 33%.

24. The method for improving the productivity of biosynthetic superoxide dismutase (SOD) according to claim 21, wherein in step (6), the superoxide dismutase (SOD) is an intracellular enzyme, and the superoxide dismutase (SOD) is a copper-and zinc-containing superoxide dismutase (SOD).

25. A method for enhancing the production rate of biosynthetic superoxide dismutase (SOD), characterized by the following steps:
(1) adding sterilized sugar 80 g/L, peptone 1.5 g/L, yeast powder 2.5 g/L, urea 1.25 g/L, copper sulfate pentahydrate 0.05 g/L, zinc sulfate heptahydrate 0.05 g/L, dipotassium hydrogen phosphate 2 g/L, potassium dihydrogen phosphate 5 g/L, magnesium sulfate heptahydrate 0.5 g/L to the fermentation tank, and then adding ultrapure water to 2.5 liters to obtain the basic medium, and
(2) inoculating the basic medium with high yeast inoculation concentration of 10-15 g/L, and
(3) setting the air intake volume of the fermentation tank to 1-1.5 v/v/m, the stirring speed to 200-500 rpm, the temperature to 28-36 °C, and the pH value to 4.5-7.5, and initiating the biosynthesis of superoxide dismutase (SOD), and
(4) when reach the initial stage of biosynthesis, which lasts for 2-6 hours, starting to continuously add carbon sources to the fermentation tank: glucose solution at 10 ml/h, with a glucose content of 50% in the glucose solution; and nitrogen sources: yeast powder solution at 0.7 ml/h, with a yeast powder content of 20% in the yeast powder solution, nitrogen peptone solution at 0.4 ml/h, with a nitrogen peptone content of 20% in the nitrogen peptone solution, and urea solution at 0.36 ml/h, with a urea content of 20% in the urea solution, and stop adding at 42-47 hours, and
(5) when reaching the middle stage of biosynthesis, which lasts for 20-24 hours, starting to continuously add 0.03 ml/h of copper sulfate pentahydrate solution with a copper sulfate content of 15%, and 0.03 ml/h of zinc sulfate heptahydrate solution with a zinc sulfate content of 15%, increasing the air flow rate to 2.5 vvm and the rotation speed to 800 rpm, and continue adding until 46-49 hours, and
(6) after the biosynthesis is completed, the biosynthesis completion time is 52-55 hours; the high yeast content obtained in the biosynthetic liquid is 20-23.6% (w/v); and the high superoxide dismutase (SOD) biological activity content is 8000-9800 U/g yeast; the production rate of yeast biosynthetic superoxide dismutase is 40029 U/L/h; wherein the calculation of high yeast content obtained in the biosynthetic liquid is based on the weight ratio (w/v) of pure wet yeast in the volume of biosynthetic liquid; the superoxide dismutase (SOD) biological activity content is calculated based on the superoxide dismutase (SOD) activity content in wet yeast, and the dry matter content in wet yeast is calculated at 33%, with the determination method being pyrogallol method; the production rate of yeast biosynthetic superoxide dismutase is calculated as the superoxide dismutase (U) biosynthesized per unit volume fermentation tank (liter) per unit time (hour).

26. The method according to claim 25, wherein in step (1), the yeast comprises Saccharomyces cerevisiae and Pichia pastoris.

27. The method according to claim 25, wherein in step (5), the wet yeast content in the biosynthetic liquid is not less than 170 g/L when the biosynthetic process reaches the middle stage, and the dry matter content of wet yeast is calculated at 33%.

28. The method according to claim 25, characterized in step (6), the superoxide dismutase (SOD) is an intracellular enzyme, and the superoxide dismutase (SOD) is a copper and zinc superoxide dismutase (SOD).
